(19) United States
(12) Patent Application Publication
(10) Pub. No.: US 2004/0094635 A1
(43) Pub. Date: May 20, 2004

(54) Air fresheners

(76) Inventors: Michael Harris, Norfolk (GB); Paul Hannington, Norfolk (GB); Clare Ewin, Norfolk (GB)

Correspondence Address: BLANK ROME LLP, 600 NEW HAMPSHIRE AVENUE, N.W., WASHINGTON, DC, 20037, US

(21) Appl. No.: 10/468,772

(22) PCT Filed: Feb. 21, 2002
(86) PCT No.: PCT/GB02/00760

(30) Foreign Application Priority Data
Feb. 21, 2001 (GB) 0104283.7
Feb. 21, 2001 (GB) 0104288.6
Feb. 21, 2001 (GB) 0104286.0

(51) Int. Cl. A61L 9/04

(52) U.S. Cl. 239/60; 424/76.4

(57) ABSTRACT

An air freshener having a fragrance releasing gel 40 having a fragrance component which is released from the composition over a sustained period of time, the gel being substantially self-supporting. The gel comprises from about 40 to about 90% by weight of the total composition of fragrance component, does not exhibit syneresis and, in use, shrinks unidirectionally away from an exposed major surface 46 when supported in a container 30′.

The present invention relates to an air freshener and in particular to substantially solid compositions for releasing fragrance or perfume into the ambient atmosphere over a prolonged period of time.

Water based gels of carrageenans have been used to carry a fragrance which is slowly released into the atmosphere. A drawback of these systems is that they carry a relatively low amount of the fragrance, typically 5% by weight, and the product will craze or crack over time as the fragrance is released and the remaining material shrinks. Water based gels can also suffer from syneresis.

There are many disclosures of gels based on a major proportion of water, such as in GB-A-938039 which uses an alginate gelling agent, EP-A-901794 which uses aluminium stearate and a polymer such as a polyamide. Other disclosures include GB-A-2 297 909 and WO 96/24389 which disclose an aqueous system with less than 20 at % fragrance, and U.S. Pat. No. 4,891,388 discloses a free standing composition in which 5 to 25% by weight of fragrance is held with a PVA (polyvinylalcohol) polymer matrix in a water/ethanol solution. The “dispenser” shrinks during use, i.e. as the fragrance evaporates.

WO 98/17243 describes a variety of uses for a novel ester terminated polyamide including the formulation of fragrance containing gels. The majority of gels disclosed use a major proportion of polyamide mixed with a C₁₃-C₁₄ iso paraffin and 25% by weight or less of fragrance. A composition having 80% fragrance and 20% polyamide is disclosed, but we have found that this exhibits syneresis (vide hereinafter).

WO 96/19247 discloses a high fragrance containing self-supporting gel composition with a dibenzylidene alditol gelling agent, and discusses the problems of syneresis in such systems and non-uniform release of the fragrance.

U.S. Pat. No. 5,780,527 describes a system in which a polymer is cross-linked in the presence of a fragrance to form a three dimensional network which contains the fragrance. These systems can contain 70 to 90% by weight of the fragrance. The preferred polymers are polyolefins, particularly maleinised polybutadiene and maleinised polyisoprene cross-linked with an ethoxylated molecule. Such products are typically quite brittle and will shrink towards a central point. This, again, provides an unsightly appearance as the gel shrinks away from the sides of the container.

GB-A-2 298 841 discloses a gel housed in an inverted container with a restricted neck or outlet. As the gel shrinks in the region of the neck, it moves down to project out of the container, into an airflow.

In U.S. Pat. No. 5,060,858, a physical anchor is provided at a container opening to anchor the gel to the edge of the opening. As the gel shrinks, it is held across the container opening by the anchor.

In U.S. Pat. No. 5,460,787 a card shaped fragrance is constructed of fragrance and a thermoplastic and then exposed on both sides. The fragrance occupies pockets formed within the continuous thermoplastic matrix.

WO 00/24434 describes a system in which the fragrance releasing composition is carried in a narrow recess, apparently in order to provide a more even release rate over the effective life of the product. This is attractive, but the use of channels reduces the surface area available to evaporation, and the product shrinks away from the channel walls during use, marring the appearance of the product. WO 00/24434 mentions the polyolefin systems referred to above, but also mentions alginates, carrageenans, and a variety of other polymers including polyamides, as the carrier matrix.

In one aspect the present invention provides an air freshener composition comprising a fragrance component, and a wax or a polymer dissolved in the fragrance component. The amount of wax or polymer dissolved in the fragrance component is sufficient to form a gel like consistency. The composition is substantially self-supporting.

The present invention also provides an air freshener comprising a composition which is self-supporting and which substantially retains its shape during the useful lifetime of the air freshener.

The prior art gel compositions are typically housed in a container with a restricted view. In part this is because the compositions crack or craze as the fragrance evaporates, becoming unsightly.

Another aspect of the present invention provides an air freshener comprising a container which is open at one end, a fragrance containing composition contained in the container and having a surface exposed to the atmosphere at the one end, wherein the exposed surface of the composition is substantially uninterrupted.

By providing an uninterrupted exposed surface, a greater surface area of the composition is open to atmosphere for a given container size, enabling a greater rate of evaporation of the solvent.

Very preferably the composition will shrink away from the exposed surface only, as the fragrance evaporates, and will stay in contact with side walls of the container.

A suitable composition can be prepared by dissolving a wax or a polymer in a liquid fragrance, to form a gel.

Again it is preferable that the composition is substantially self-supporting. In particular the container may be stood with the exposed face of the composition substantially vertical.

Yet another aspect of the present invention provides an air freshener comprising a gel-like or solid, fragrance containing, composition which is exposed to the atmosphere on opposite surfaces. The composition may be moulded to a substantially flat shape and is surrounded by a peripheral container wall, leaving opposed major surfaces exposed to the atmosphere. Preferably one or both of the exposed surfaces is substantially uninterrupted.

Preferably, during evaporation of the fragrance, the composition shrinks only in a direction perpendicular to the exposed surface and so remains in contact with the peripheral wall. A preferred composition is formed by dissolving a wax or a polymer in a fragrance.

We have found that by appropriate selection of the fragrance and the wax or polymer, it is possible to achieve a composition having a high fragrance content, 50% by weight or more. Such a high fragrance containing composition has improved performance characteristics such as giving a greater impact in-use or maintaining a more even release of fragrance over the product lifetime. More particularly, the composition shrinks one dimensionally as the fragrance evaporates, i.e. the product only shrinks away from the exposed surfaces.

Thus, the composition may be shaped in a mould, and will retain the shape during use, when the fragrance evaporates.

If the dissolved wax or polymer content or other gel forming agent is too high, the available fragrance is reduced, and also the product is likely to be harder or more brittle and so may have less desirable shrinkage characteristics as the fragrance evaporates. The gel forming agent to fragrance ratio can be optimised by trial and error to meet the desired performance characteristics.

The composition preferably comprises more than about 50% by weight of fragrance component, more preferably more than about 70% by weight of fragrance component.

Very preferably the composition has from about 70% to about 90% by weight of fragrance component, more preferably from about 75% to about 85% by weight.

The fragrance component is a derivative of a liquid hydrocarbon. It may be a discrete chemical but more typically will be a complex mixture of volatile liquid ingredients of natural or synthetic origin. The fragrance component may be presented in an oily carrier liquid, typically 50% fragrance and 50% carrier. We prefer a fragrance having a high fragrance content, i.e. little or no oily carrier, as this maximises the fragrance available for evaporation in use.

The wax or polymer should be matched to the fragrance component to achieve the desired degree of solubility in the fragrance component. If there is a mismatch, the polymer may form a cloudy solution or gel: although this is not necessarily undesirable a clear product is usually preferred. Waxes tend to give an opaque appearance both before and after mixing with a fragrance. More importantly, a higher wax or polymer content, and hence a reduced fragrance content, may be required to achieve a sufficiently self supporting product.

Fragrance components with low polarity molecules are generally preferred. The fragrance should also have a relatively well defined working vapour pressure to provide the necessary evaporation at ambient temperatures.

Functional groups on the polymer structure will also affect the solubility of the polymer in the fragrance component.

Very preferably the, or the main, polymer component is a polyamide polymer. A particularly preferred polyamide is supplied as UNICLEAR 100 from Arizona Chemical Co., USA, which is solid at room temperature. UNICLEAR 80V, which is from a vegetable source and incorporates 20% mineral oil is also a preferred polyamide. Such ester terminated polyamide polymers are described in WO98/17105.

Another preferred polymer is a styrene based polymer such as a styrene block copolymer

A wax, a high molecular weight hydrocarbon, may also be used to form the gel.

Another formulation uses a stearate to form the gel.

The composition may include a variety of additives as commonly used in the art, including inert additives such as flowers or beads for aesthetic purposes, soluble additives such as colourants, or dispersed additives such as pearlescent particles, glitters, metallic pigments or thermochromic pigments, photochromic agents, optical brightener agents.

A composition or formulation in accordance with the invention may be prepared by warming the wax or polymer and the fragrance component with gentle mixing. At an elevated temperature, typically about 65° C., the wax or polymer dissolves or disperses in the fragrance component. The warm solution is poured into containers or moulds. On cooling a single phase anhydrous gel may be formed. Depending on the vapour pressure characteristics of the fragrance, the mixing temperature should be kept as low as possible to avoid driving off too much of the fragrance components.

The compositions are adapted to be poured into a container with an open surface. The container can then be placed with the open surface vertical, and the composition adheres to the inner surface of the container, without falling out as the mixture shrinks on evaporation of the fragrance.

Preferably the composition is substantially transparent. A label or the like may be provided on the container and visible through the air freshener composition.

The mould may be shaped to allow release of the cooled solid composition to provide a free standing product.

The invention will be further described by way of example. All amounts are % by weight of the total composition.

### EXAMPLE 1

A lemon fragrance product was produced by warming UNICLEAR 100 (20% by weight), Solvent Yellow 93 colorant (0.01%) (Clariant Sandoplast Yellow 3G) and Orange Turpene fragrance (balance %) to 65° C. and mixing gently until a clear liquid was formed. The liquid was then poured into glass moulds and allowed to cool.

The cooled product had the following characteristics:

1. Shrinkage, due to fragrance evaporation, was one dimensional, i.e. the moulded product when exposed on one surface only became thinner with evaporation.

2. There was extended, slow release of fragrance, over a period of more than two weeks.

3. Transparency was maintained through the effective life of the product—the polymer did not precipitate out.

4. The composition adhered well to the sides of the mould, even when inverted.

### EXAMPLE 2

The following composition was prepared as in Example 1.

| | % W/W |
|---|---|
| UNICLEAR 100 | 50 |
| * Solvent Red 27 | 0.01 |
| * Solvent Blue 35 | 0.01 |
| French Lavender Oil | Balance |

* Clariant Fat-Red 5B02 and Clariant Fat Blue B01.

The cooled product had properties similar to those described in Example 1.

### EXAMPLES 3 TO 7

The following compositions were prepared as for Example 1. The percentage loss (evaporation) of the fragrance was then measured

| Component/Ex | 3 | 4 | 5 | 6 | 7 |
|---|---|---|---|---|---|
| UNICLEAR 100 | 25 | 25 | 25 | 25 | 25 |
| Fragrance PF30551 | | | 75 | | |
| Fragrance PF30549 | | | | 75 | |
| Fragrance PF30550 | | | | | 75 |
| Fragrance PF30552 | | 75 | | | |
| Fragrance PF30548 | 75 | | | | |
| Dye | q.s | q.s | q.s | q.s | q.s |

All fragrances were supplied by Phoenix Fragrance. UNICLEAR 100 was supplied by Arizona Chemical Co, USA.

A blend of 80% Polyamide, 20% Mineral Oil, such as UNICLEAR 80V was also used. It is thought that the blending of the polyamide with the mineral oil, in UNTICLEAR 80V, may help to promote dissolution of the polymer in the fragrance. The polymer is a hard waxy polymer melting at 90° C., but it will ‘melt’ at about 65° C. in the presence of the fragrance.

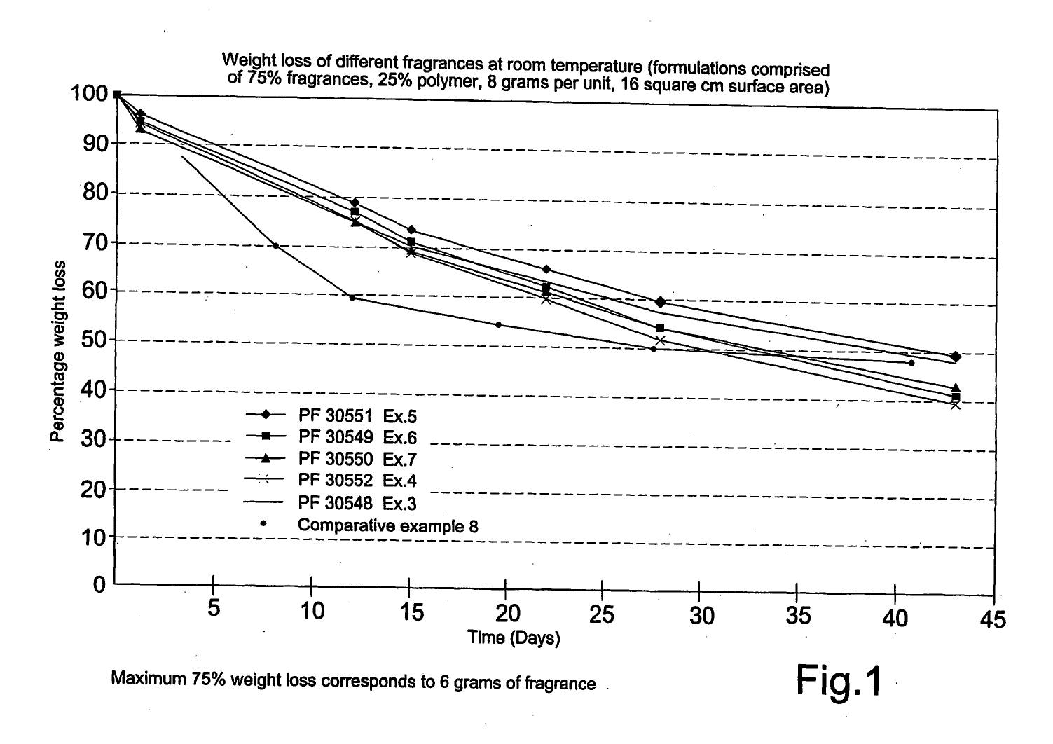

8 gm of the warm polymer/fragrance mix was poured into a glass mould having an exposed upper surface of 16 cm², giving a depth of about 5 mm. The weight loss equates to the amount of volatile fragrance components which evaporate. This was measured over time, and is presented in FIG. 1.

It can be seen that after an initial period of one or two days, the rate of evaporation of fragrance is substantially linear for an extended period of time. This provides for a product having substantially even performance for an extended period, four weeks or more.

### COMPARATIVE EXAMPLE 8

By way of comparison a similar test was performed on a commercial product, HAZE CRYSTAL AIR EXOTIC FRUITS manufactured by Reckitt Benckiser which is believed to made in accordance with WO 00/24434. This shows a steeper initial fragrance loss, but then a much lower rate of fragrance evaporation after about 10 days.

### EXAMPLE 9

Perfume PF30551, 65% by weight, was mixed with KRATON 1652 (ex Shell Chemicals) 65% by weight and heated to 100° C. The KRATON polymer dissolved in the perfume and the mix was then poured into a shallow mould and allowed to cool to a gel. The gel was not as solid as Examples 1 to 7, but good fragrance release characteristics were obtained.

It will be appreciated that a fragrance with a flash point above the mixing temperature should be chosen.

KRATON is a styrenic block co-polymer. Such polymers are produced by polymerising styrene and then sequentially reacting with butadiene or isoprene to produce linear A-B-A, radial (A-B)n or di-block (A-B) polymers as required.

### EXAMPLES 10 to 15

Waxes were also dissolved in a fragrance composition to produce a solid gel formulation. An opaque product is formed with a hard gel. Some are subject to cracking as the perfume evaporates but good performance with prolonged perfume release over several weeks is obtained.

| | | | % | |
|---|---|---|---|---|
| Ex. | Wax | % Wax | Perfume⁷ | Observations |
| 10 | Hydrogenated | 50 | 50 | Brittle and |
| | Castor Oil¹ | | | cracks on |
| | | | | shrinkage |
| 11 | Polyglyceryl-3 | 28 | 72 | Brittle and |
| | Beeswax² | | | cracks on |
| | | | | shrinkage |
| 12 | Beeswax³ | 43 | 57 | No cracking |
| 13 | Microcrystalline | 18.5 | 81.5 | No cracking |
| | Wax⁴ | | | |
| 14 | Paraffin Wax⁵ | 35 | 65 | No cracking |
| 15 | Carnauba Wax⁶ | 46.4 | 53.4 | No cracking |

¹Liowax PM80 from Miracema-Nuodex
²Cera Bellina from Jan Dekker
³White Beeswax BP from Poth Hille
⁴Microcrystalline Wax 3749 from Poth Hille
⁵Paraffin Wax 125/130 from Astor Stag
⁶Carnauba Wax from Stanley Black
⁷Orange Turpenes

### EXAMPLE 16

A stearate based formulation was prepared as follows. 5.0% sodium stearate, 5.0% water, 10.0% ethanol and 80.0% fragrance were mixed at 80° C. until homogenous. The mixture was then passed into a mould and allowed to cool

The cooled formulation adhered to a glass mould.

### COMPARATIVE EXAMPLE 17

A mixture of 20% UNICLEAR 100 V and 80% d-limonene fragrance was warmed to about 65° C. and mixed to form a homogenous mixture. The cooled mixture formed a self-supporting block but exhibited syneresis at room temperature: fragrance oozed or leaked from the lower region of the block. The cooled block was also brittle.

FIG. 1 is a plot of weight loss over time for example 3 to 7 above;

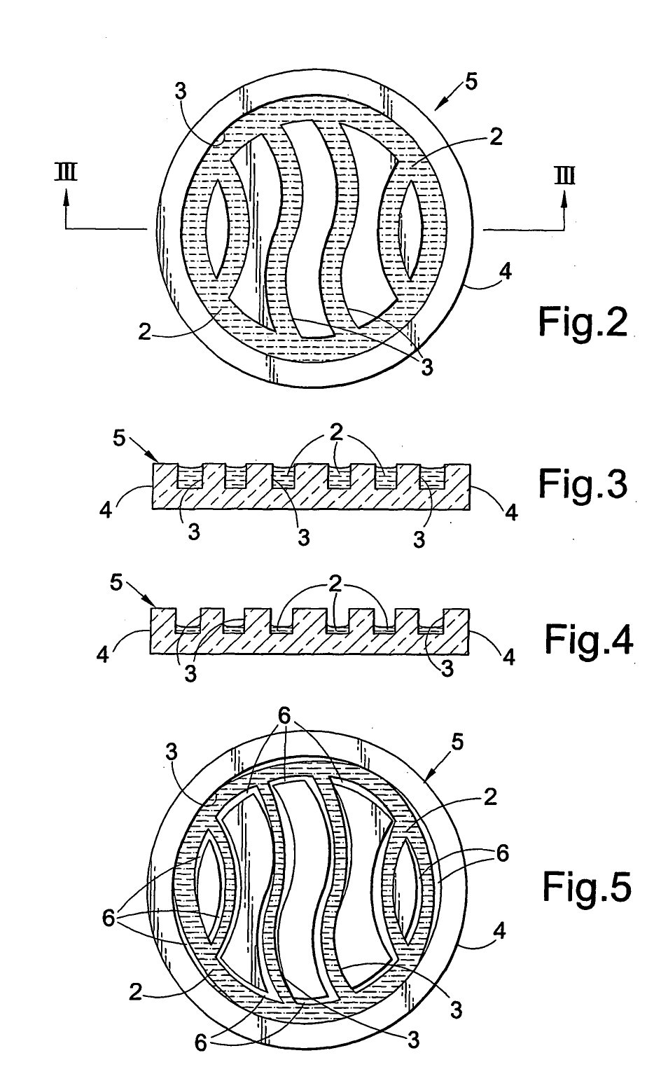

FIG. 2 shows a plan view of an air freshener product containing a composition in accordance with the invention;

FIG. 3 is a cross-section on line III-III of FIG. 2;

FIG. 4 is similar to FIG. 3, but showing the product part used;

FIG. 5 shows a plan view of a prior art product after use;

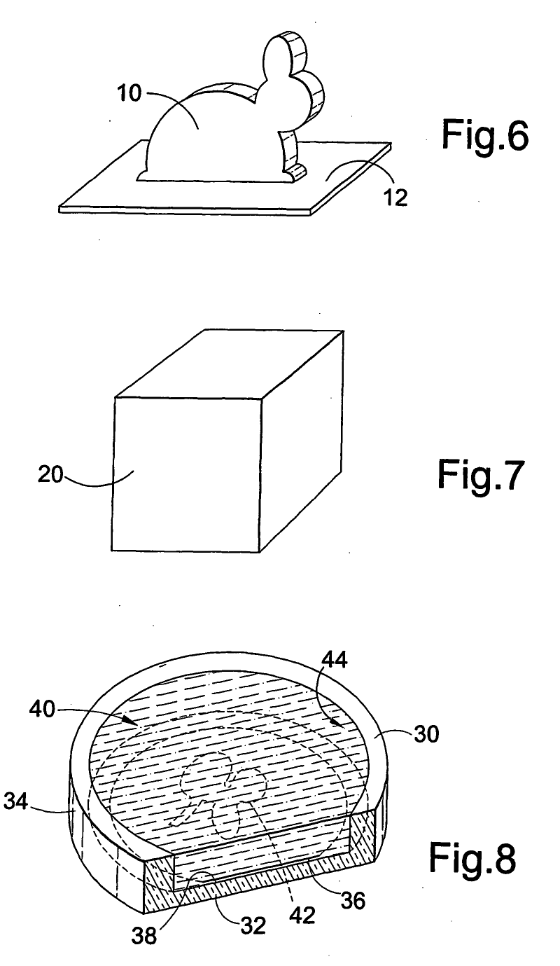

FIG. 6 shows an air freshener product forming a second embodiment of the invention;

FIG. 7 shows an air freshener product forming a third embodiment of the invention;

FIG. 8 shows a perspective view partly cut away of a fourth embodiment of the invention.

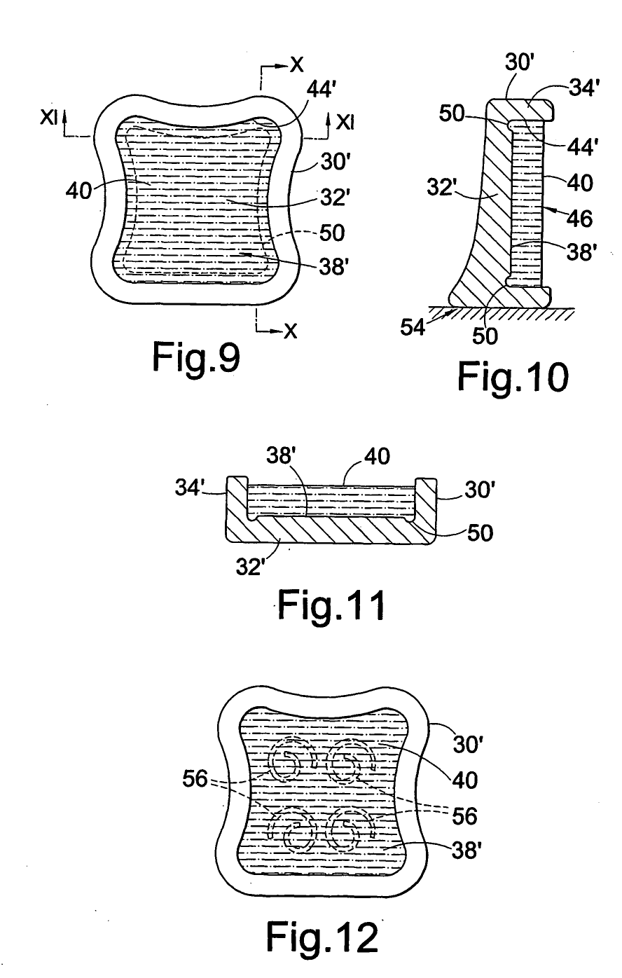

FIG. 9 is a plan view of a fifth embodiment of the invention, similar to the embodiment of FIG. 8,

FIG. 10 is a cross-section along lines X-X of FIG. 9,

FIG. 11 is a cross-section along lines XI-XI of FIG. 9,

FIG. 12 illustrates a modification of the embodiment of FIGS. 9 to 11.

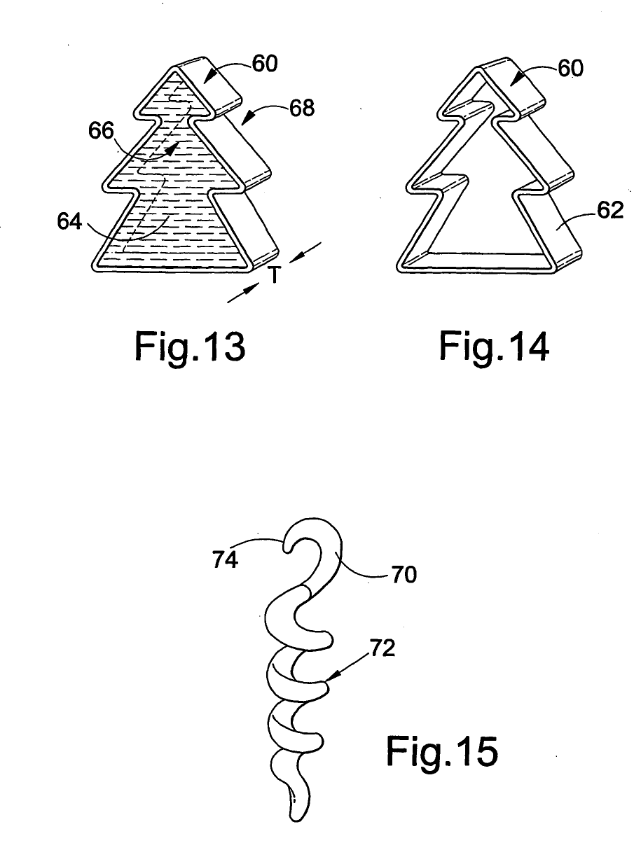

FIG. 13 shows a perspective view of yet another embodiment of the invention, and

FIG. 14 shows a container of the embodiment of FIG. 13.

FIG. 15 shows yet another embodiment of the invention.

FIG. 2 shows a plan view of an air freshener comprising a composition 2 prepared in accordance with Example 3 above, and contained in a glass mould 4. The composition 2 fills channels 3 provided in a major surface 5 of the mould 4. As seen in FIG. 3, the channels 3 are initially filled with the composition 2. The cross-section of FIG. 4 shows the product after exposure to the ambient atmosphere for about three weeks. Fragrance has evaporated, causing apparent shrinkage of the remaining composition into the channels 3. It can be seen that the composition shrinks one-dimensionally, that is it continues to fill the channels 3 across their full width. Hence the plan view is still as seen in FIG. 2. If the product is left until substantially all the fragrance is evaporated, the polymer component will remain in the bottom of the channels 3.

FIG. 5 shows a plan view of the composition of comparative example 8 after about three weeks. It can be seen that the remaining composition has shrunk away from the sides of the channels 3, forming gaps 6 and giving a quite different visual appearance, which is uncontrolled during the life of the product.

FIG. 6 shows a second embodiment of the invention. A shape 10, in this case a rabbit shape about 5 cm high and 1.5 cm thick is formed by casting the molten composition of one of Examples 1 to 7 or 9 to 16 in a mould. The mould may be coated with a release agent to allow release of the shape, but the release agent should not contaminate the surface of the finished product, or otherwise inhibit evaporation of the fragrance component. The gel is self-supporting. In practice the shape may be supplied on a base 12 to protect any supporting surface from the oils etc. in the composition. Components having little or no skin hazards are preferred because of the likelihood of handling by the user.

FIG. 7 shows a third embodiment of the invention, simple cube shape 20, formed with a composition according to one of Examples 1 to 7 or 9 to 16. The initial cube has a side of about 10 cm, and a fragrance content of about 75%. After loss of the fragrance, the cube will have shrunk to about 25% of its volume, i.e. about 6 cm on a side.

FIG. 8 shows a perspective view of a fourth embodiment of the invention. A glass container 30 has a bottom wall 32 which is flat and a peripheral wall 34. The container has an internal depth of about 6 mm. A label 36, which may carry a logo, flower design 42, etc., is stuck to the inner surface 38 of bottom wall 32. A transparent composition prepared in accordance with one of Examples 1 to 7 is poured into the container 30 while molten. The composition cools to a clear gel 40 which adheres well to the inner surface 44 of the side wall 34 and the label 36. (If preferred, the label could be provided on the underside of wall 32 and be visible through the wall). As the fragrance evaporates, the gel becomes thinner, but does not shrink away from the side wall 34, and hence continues to provide an attractive appearance covering the label 36. Printing 42 on the surface of label 36 can be seen through the gel 40.

Referring to FIG. 9, a glass container 30′ is provided with a peripheral groove 50 at the junction between the inner wall surface 44′ and the bottom wall 32′. This forms an anchor which helps to maintain the gel 40 flat against the surface 38′ of the bottom wall 32′ of the container 30′. The wall 32′ is thicker towards one bottom side 54, to provide a wider edge on which the container 32′ can stand so that the exposed gel surface 46 is vertical. In FIG. 12, grooves 56 are provided in the form of a pattern in surface 38′ of bottom wall 32′. Again these provide an anchor, but they will also provide an attractive effect. The increased depth of the transparent or translucent gel at the groove 56 reduces light transmission and results in a darker area, a pattern being formed at the groove.

It will be understood that the degree of transparency or translucence of the gel can be adjusted, for example by adjusting the amount of colourant used. Examples 9 to 16 can also be used with FIGS. 8 to 12, except that the gel is opaque.

Referring to FIG. 13, another embodiment of the invention comprises a metal container 60 which is in the shape of a Christmas tree and, as seen in FIG. 14, forms only a peripheral wall 62, the major front and back surfaces being open. The container 60 is filled with a composition 64 prepared in accordance with one of Examples 1 to 7 and 9 to 16 and which is substantially self supporting and adheres well to the wall 62. The composition 64 is exposed on front and back major surfaces 66, 68. In use, as the fragrance evaporates, the composition decreases in thickness T, between the major surfaces, but remains in contact with the peripheral wall 62.

This arrangement may also be used with prior art compositions such as described in WO 00/24434. The arrangement provides the benefit of enhanced fragrance evaporation, because two major surfaces of the air freshener composition are exposed to the atmosphere. However, with the prior art compositions some shrinkage from the peripheral wall 12 is to be expected, with the composition eventually shrinking to a husk at the lower region of the container.

An exemplary prior art composition is as follows.

| Ingredient | % w/w |
|---|---|
| Carrageenan | 2.00 |
| Monopropylene Glycol | 2.00 |
| Biocide | 0.1 |
| Fragrance | 3.0 |
| Fragrance Emulsifier | 1.0 |
| Colourant | q.s |
| Soft Water | balance |

The carrageenan is mixed with the water and heated to 70° C. The glycol and dye are then added. The fragrance and emulsifier are pre-mixed and then added to the main mix. Sodium stearate may also be added to improve the fragrance solvency. The composition is then poured into the container and left to cool and set. It will be appreciated that the container is laid flat on a surface to support the composition while it cools to the setting temperature.

In the embodiment of FIG. 15, a glass spiral 70 was dipped into the composition of Example 15 above, with the composition at a temperature of about 60° C. The glass spiral was removed and a coating 72 of the composition remained on the spiral. The spiral was then re-dipped, to a total of three dips, to leave a coating of about 2 to 3 mm thickness on the glass spiral. The spiral has a hook shape 74 at its upper end for it to be hung by a thread, for example.

The embodiment of FIG. 15 may also be prepared using one of the other exemplary compositions.

1. An air freshener having a fragrance releasing gel having a fragrance component which is released from the composition over a sustained period of time, the gel being substantially self-supporting, characterised in that the gel comprises from about 40 to about 90% by weight of the total composition of fragrance component, does not exhibit syneresis and, in use, shrinks unidirectionally away from an exposed major surface when supported in a container.

2. An air freshener according to claim 1, wherein the fragrance forms a continuous phase.

3. An air freshener according to claim 1 or 2, wherein the fragrance is a derivative of a liquid hydrocarbon.

4. An air freshener according to claim 1, 2 or 3, wherein the gel is formed by a component selected from the group consisting of
(i) a polyamide polymer
(ii) a wax
(iii) a stearate
(iv) a styrene based polymer.

5. An air freshener according to claim 4, wherein when a polyamide polymer is present, the fragrance component is about 75% or less by weight of the composition, and preferably is about 40 to 60% by weight, and more preferably is about 50 to 60% by weight.

6. An air freshener according to claim 4 or 5, wherein a mineral oil component is incorporated in the polyamide polymer.

7. An air freshener as claimed in claim 4, wherein when a wax is present the fragrance component is about 50 to 80% by weight of the composition, and more preferably about 50 to 65% by weight.

8. An air freshener as claimed in claim 4, wherein when a styrene polymer is present the fragrance component is about 50 to 70% by weight of the composition, and more preferably about 50 to 60% by weight.

9. An air freshener as claimed in claim 4, wherein when a stearate is present the fragrance component is about 70 to 90% by weight of the composition, and more preferably about 75 to 85% by weight.

10. An air freshener as claimed in any one of claims 1 to 9, comprising a container which is open at one side, the fragrance releasing gel in the container and having a surface exposed to the atmosphere at the one end, the exposed surface of the composition being substantially uninterrupted.

11. An air freshener as claimed in claim 10, wherein the container is adapted to be used with the one side substantially vertical.

12. An air freshener as claimed in claim 10 or 11, wherein the container has a peripheral wall and the composition adheres to the peripheral wall in use.

13. An air freshener as claimed in any one of claims 1 to 9, comprising a container which is open at opposed sides, and the fragrance releasing gel held in the container and exposed to the atmosphere at the opposed sides of the container.

14 An air freshener as claimed in claim 13, wherein the container is adapted to stand with the opposed sides substantially vertical.

15. An air freshener as claimed in claim 13 or 14, wherein the container comprises a peripheral wall, the composition adheres in use to the peripheral wall and being exposed at front and back surfaces which are defined by the peripheral wall.

16. An air freshener as claimed in any one of claims 1 to 9, wherein the air freshener is formed into a three dimensional shape which is substantially unsupported by a container in use.

17. An air freshener as claimed in claim 16, having from 50 to 70% by weight of fragrance.
